(19) United States
(12) Patent Application Publication
(10) Pub. No.: US 2021/0205195 A1
(43) Pub. Date: Jul. 8, 2021

(54) PRESERVED COMPOSITIONS

(71) Applicant: Conopco, Inc., d/b/a UNILEVER, Englewood Cliffs, NJ (US)

(72) Inventors: Stuart CAMPBELL-LEE, Wirral (GB); Thomas Richard POINTON, Warrington (GB)

(21) Appl. No.: 17/056,618

(22) PCT Filed: May 20, 2019
(86) PCT No.: PCT/EP2019/062974

§ 371 Date: Nov. 18, 2020

(30) Foreign Application Priority Data
Jun. 4, 2018 (EP) 18175850.9

(51) Int. Cl. A61K 8/362; A61K 8/368; A61K 8/31; A61K 8/34; A61K 8/41; A61Q 19/10; A61Q 5/12

(52) U.S. Cl. CPC A61K 8/362; A61K 8/368; A61K 8/31; A61K 8/347; A61K 2800/5922; A61K 8/416; A61Q 19/10; A61Q 5/12; A61K 2800/524; A61K 8/342

(57) ABSTRACT

A composition comprising itaconic acid or salt thereof and a cationic surfactant.

### FIELD OF THE INVENTION

The present invention relates to the area of preservation for consumer goods, in particular personal care compositions.

### BACKGROUND OF THE INVENTION

In the consumer goods industry, particularly the personal care and cosmetic industry there is a need for new preservation, especially preservation chemicals that are from natural sources, that are abundant and readily available.

A large number of antimicrobial active compounds are already employed in the personal care industry, but alternatives nevertheless continue to be sought. Not all antimicrobial agents have preservative properties and thus the need for new preservation chemicals is particularly required. It is to be noted that the substances used in the personal care field must be
toxicologically acceptable
readily tolerated by the skin
stable
largely and preferably completely odourless
inexpensive to prepare (i.e. employing standard processes and/or starting from standard precursors)
easy to formulate (i.e. preferably liquid) and should not be detrimental to the final product.

The present application has found a preservation system suitable for use with consumer goods, particularly personal care compositions. The preservation system is readily available, cost effective and easy to formulate with compositions of the invention

### DESCRIPTION OF THE INVENTION

The present invention relates to an aqueous composition comprising itaconic acid or salt thereof, a cationic surfactant and at least 75 wt % water.

The invention further relates to a method of preserving an aqueous composition comprising cationic surfactant and at least 75 wt % water, by the use of itaconic acid or salt thereof.

### DETAILED DESCRIPTION OF THE INVENTION

Compositions of the invention comprise itaconic acid or salt thereof.

Preferably the total level of itaconic acid in the total composition is from 0.05 to 7 wt % % of the total composition, more preferably 0.1 to 5 w % t most preferably from 0.2 to 3 wt %.

The compositions of the invention comprise at least 75 wt %, preferably at least 80 wt %, more preferably at least 85 wt % and most preferably at least 87 wt % of water.

Preferably the composition further comprises a cyclic or acyclic, aromatic organic compound and or terpene, more preferably the cyclic or acyclic compound comprises an aldehydes, ketones, amides, amine, alcohol, and ester group. Preferably the level of cyclic or acyclic organic compound is from 0.05 to 3 wt % of the total composition

In one preferred embodiment the cyclic or acyclic organic compound comprises an aromatic compound, preferably the aromatic compound is benzoic acid or salt thereof.

In a second preferred embodiment the composition further comprises a terpene or thymol, a particularly preferred terpene is terpineol.

In a further embodiment the composition further comprises a cyclohexane.

Preferably the individual level of aromatic compound, benzoic acid or salt thereof, thymol or terpene or cyclohexane is from 0.05 to 3 wt % of the total composition

Compositions of the invention comprise cationic surfactants preferably comprising amino or quaternary ammonium hydrophilic moieties which are positively charged when dissolved in an aqueous composition.

The method of the invention is particularly suitable for the production of samples of conditioning compositions (such as hair conditioners) comprising structured liquid phases. The structured liquid phases of hair conditioners are usually characterized as lamellar gel networks formed from cationic surfactant and fatty alcohol bilayers. The bilayers may grow, swell or fold to form extended sheets or spherical vesicles.

The method according to the invention is most preferably used to produce samples of hair conditioners comprising lamellar gel networks formed from cationic surfactant and fatty alcohol bilayers.

Examples of suitable cationic surfactants which are preferred include quaternary ammonium cationic surfactants corresponding to the following general formula:

[N(R1)(R2)(R3)(R4)]+ (X)−

in which R1, R2, R3, and R4 are each independently selected from (a) an aliphatic group of from 1 to 22 carbon atoms, or

(b) an aromatic, alkoxy, polyoxyalkylene, alkylamido, hydroxyalkyl, aryl or alkylaryl group having up to 22 carbon atoms; and X is a salt-forming anion such as those selected from halide, (e.g. chloride, bromide), acetate, citrate, lactate, glycolate, phosphate nitrate, sulphate, and alkylsulphate radicals.

The aliphatic groups can contain, in addition to carbon and

hydrogen atoms, ether linkages, and other groups such as amino groups. The longer chain aliphatic groups, e.g., those of about 12 carbons, or higher, can be saturated or unsaturated.

Specific examples of such quaternary ammonium cationic surfactants of the above general formula are cetyltrimethylammonium chloride, behentrimonium chloride, behentrimonium chloride (BTAC), cetylpyridinium chloride, cetrimonium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, dipalmitoylethyldimethylammonium chloride, PEG-2 oleylammonium chloride and salts of these, where the chloride is replaced by other halide (e.g., bromide), acetate, citrate, lactate, glycolate, phosphate nitrate, sulphate, or alkylsulphate.

In a preferred class of cationic surfactant of the above general formula, R1 is a C₁₆ to C₂₂ saturated or unsaturated, preferably saturated, alkyl chain and R², R³ and R⁴ are each independently selected from CH₃ and CH₂CH₂OH, preferably CH₃.

Specific examples of such preferred quaternary ammonium cationic surfactants are cetyltrimethylammonium chloride (CTAC), behentrimonium chloride (BTAC) and mixtures thereof.

Alternatively, primary, secondary or tertiary fatty amines may be used in combination with an acid to provide a cationic surfactant suitable for use in the invention. The acid protonates the amine and forms an amine salt in situ. The amine is therefore effectively a non-permanent quaternary ammonium or pseudo-quaternary ammonium cationic surfactant.

Suitable fatty amines of this type include amidoamines of the following general formula:

R¹—C(O)—N(H)—R²—N(R³)(R⁴)

in which R¹ is a fatty acid chain containing from 12 to 22 carbon atoms, R² is an alkylene group containing from one to four carbon atoms, and R³ and R⁴ are each independently, an alkyl group having from one to four carbon atoms.

Specific examples of suitable materials of the above general formula are stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, and diethylaminoethylstearamide.

Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidyl behenylamine.

Particularly preferred is stearamidopropyldimethylamine.

In a preferred embodiment the cationic is present as a gel conditioning phase having a lamellar structure.

The level of cationic surfactant is preferably from 0.01 to 10, more preferably 0.05 to 5, most referably 0.1 to 2 w.t. % of the total composition.

Preferably the weight ratio of cationic surfactant to unsaturated organic acid, preferably itaconic acid is from 1:1-1:100, more preferably from 1:1 to 1:20.

A further acid used may be used to protonate the amine. Suitable acids include hydrochloric acid, citric acid, acetic acid, tartaric acid, fumaric acid, lactic acid, malic acid, succinic acid, and mixtures thereof. Preferably, the acid is selected from the group consisting of acetic acid, tartaric acid, hydrochloric acid, lactic acid and mixtures thereof.

Mixtures of any of the above-described cationic surfactants may also be suitable.

Examples of suitable fatty alcohols which are useful in the invention include fatty alcohols with a melting point of 25° C. or higher. Generally, the melting point ranges from 25° C. up to 90° C., preferably from 40° C. up to 70° C. and more preferably from 50° C. up to about 65° C.

The fatty alcohol can be used as a single compound or as a blend or mixture of at least two fatty alcohols. When a blend or mixture of fatty alcohols is used, the melting point means the melting point of the blend or mixture.

Suitable fatty alcohols of this type have the general formula CH₃(CH₂)ₙOH, where n is an integer from 7 to 29, preferably from 15 to 21.

Specific examples of suitable fatty alcohols are cetyl alcohol, cetearyl alcohol, stearyl alcohol, behenyl alcohol, and mixtures thereof. Cetyl alcohol, cetearyl alcohol, stearyl alcohol and mixtures thereof are particularly preferred.

Mixtures of any of the above-described fatty compounds may also be used.

Preferred compositions of the invention are preferably personal care compositions. More preferably hair care compositions, in particular conditioner compositions, most preferably rinse off compositions.

Preferred hair treatment compositions are selected from a rinse-off hair conditioner and a hair mask.

Rinse off conditioners for use in the invention are conditioners that are typically left on wet hair for 1 to 2 minutes before being rinsed off.

Hair masks for use in the present invention are treatments that are typically left on the hair for 3 to 10 minutes, preferably from 3 to 5 minutes, more preferably 4 to 5 minutes, before being rinsed off.

A preferred conditioner comprises a conditioning gel phase. Such conditioners and methods for making them are described in WO2014/016354, WO2014/016353, WO2012/016352 and WO2014/016351. A preferred hair conditioning composition of this type comprises from 0.4 to 8% wt. fatty alcohol having from 8-22 carbons, from 0.1 to 2 wt % cationic surfactant component, water, and wherein the composition confers a Draw Mass of from 1 to 250 g to hair treated with the conditioning composition. Draw Mass is the mass required to draw a hair switch through a comb or brush.

The conditioning compositions may also comprise other optional ingredients. Such ingredients include, but are not limited to; fatty material, deposition polymers and further conditioning agents.

Conditioner compositions preferably additionally comprise fatty materials. The combined use of fatty materials and cationic surfactants in conditioning compositions is believed to be especially advantageous, because this leads to the formation of a structured lamellar or liquid crystal phase, in which the cationic surfactant is dispersed.

By “fatty material” is meant a fatty alcohol, an alkoxylated fatty alcohol, a fatty acid or a mixture thereof.

Preferably, the alkyl chain of the fatty material is fully saturated.

Representative fatty materials comprise from 8 to 22 carbon atoms, more preferably 16 to 22. Examples of suitable fatty alcohols include cetyl alcohol, stearyl alcohol and mixtures thereof. The use of these materials is also advantageous in that they contribute to the overall conditioning properties of compositions.

Alkoxylated, (e.g. ethoxylated or propoxylated) fatty alcohols having from about 12 to about 18 carbon atoms in the alkyl chain can be used in place of, or in addition to, the fatty alcohols themselves. Suitable examples include ethylene glycol cetyl ether, polyoxyethylene (2) stearyl ether, polyoxyethylene (4) cetyl ether, and mixtures thereof.

The level of fatty material in conditioners is suitably from 0.01 to 15, preferably from 0.1 to 10, and more preferably from 0.1 to 5 percent by weight of the total composition. The weight ratio of cationic surfactant to fatty alcohol is suitably from 10:1 to 1:10, preferably from 4:1 to 1:8, optimally from 1:1 to 1:7, for example 1:3.

Further conditioning ingredients include esters of fatty alcohol and fatty acids, such as cetyl palmitate.

A conditioning composition for use in the present invention may comprise a micellar structured liquid.

The pH of a conditioner comprising the present composition is preferably 3-5.5. More preferably the pH of the composition is 4.0-5.0.

Where the composition has a pH of less than 3.10 it is preferred that it is in the form of a conditioning mask for intense treatment.

Further conditioning ingredients include conditioning oils, preferably selected from coconut oil and olive oil.

The compositions of the invention may contain, emulsified droplets of a silicone conditioning agent, for enhancing conditioning performance.

Suitable silicones include polydiorganosiloxanes, in particular polydimethylsiloxanes which have the CTFA designation dimethicone. Also suitable for use compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188.

A further preferred class of silicones for inclusion in compositions according the invention, in particular shampoos and conditioners of the invention are amino functional silicones. By “amino functional silicone” is meant a silicone containing at least one primary, secondary or tertiary amine group, or a quaternary ammonium group. Examples of suitable amino functional silicones include: polysiloxanes having the CTFA designation “amodimethicone”.

Specific examples of amino functional silicones suitable for use in the invention are the aminosilicone oils DC2-8220, DC2-8166 and DC2-8566 (all ex Dow Corning).

Suitable quaternary silicone polymers are described in EP-A-0 530 974. A preferred quaternary silicone polymer is K3474, ex Goldschmidt.

Also suitable are emulsions of amino functional silicone oils with non ionic and/or cationic surfactant.

Pre-formed emulsions of amino functional silicone are also available from suppliers of silicone oils such as Dow Corning and General Electric. Specific examples include DC939

Cationic Emulsion and the non-ionic emulsions DC2-7224, DC2-8467, DC2-8177 and DC2-8154 (all ex Dow Corning).

The total amount of silicone is preferably from 0.01 wt % to 10 wt % of the total composition more preferably from 0.1 wt % to 5 wt %, most preferably 0.5 wt % to 3 wt % is a suitable level.

The invention will now be illustrated by the following non-limiting Examples

### Examples

A conditioner formulation according to table 1 was prepared.

TABLE 1

| | Percentage |
|---|---|
| Ingredient | active |
| Cetostearyl alcohol | 5.000 |
| Stearamidopropyl dimethylamine | 1.250 |
| BTAC | 0.875 |
| Silicone emulsion | 3.000 |
| Water and minors | To 100.000 |

Itaconic acid, lactic acid and citric acid were added the conditioner composition at 0.5 wt %. and 1 wt % of the total level of conditioner.

Sample Preparation

Itaconic acid, lactic acid and citric acid were purchased from Sigma-Aldrich. Six samples of unpreserved conditioner were dosed with 0.5% and 1% (w/w) itaconic acid, citric acid and lactic acid separately. An unpreserved sample was kept as a control. Each dosed product was adjusted to pH 5 using 50% sodium hydroxide (Sigma-Aldrich).

Challenge Test Method

A modified challenge test to that of the European Pharmacopoeia (Ph. Eur. Or EP) 5.1.3 criteria was performed on a subsample of unpreserved and each dosed product. The bacterial challenge test pool and inoculum level are summarized in table 2. The microbial challenge pools were added to each sample container at a ratio of 1:100. The final concentration of inoculum in the product should be 5×10⁶ CFU/G of test product. Each product is mixed with a sterile spatula to ensure a homogenous distribution of the inocula throughout the product.

TABLE 2

| | | | Pool | In-product |
|---|---|---|---|---|
| | | | Inoculum | inoculum |
| Pool | Group | Microorganism | CFU/mL | CFU/G |
| 1 | Gram | Pseudomonas aeruginosa | 7.1 × 10⁸ | 5 × 10⁵⁻⁶ |
| | Negative non- | Burkholderia cepacia | CFU/mL | CFU/mL |
| | fermenters | Strenotrophomonas sp | | |
| 2 | Gram | Enterobacter cloacae | 5.2 × 10⁸ | 5 × 10⁵⁻⁶ |
| | Negative | Klebsiella sp | CFU/mL | CFU/mL |
| | fermenters | | | |

Both the inoculum level and the level of microorganism within each sample was quantified using a Total Viable Count (TVC) pour plate method at 7, 14, 21 and 28 days. A 1:10 dilution was made with a subsample of each product, performed separately in Peptone (0.1%)/tween 80 (2%) neutralising agent. A 1:10 and 1:100 dilution of each subsample was performed and pour plates produced at each dilution using tryptone soya agar. Plates were incubated at 28° C. for 48 hours and then examined for growth. Visible colonies were counted with the aid of a Quebec Colony Counter and recorded for analysis against the challenge test criteria. During the removal of a subsample of product for TVC, a reinoculation is performed at 7 and 14 days, reintroducing 5×10⁶ CFU/G of bacteria before mixing using a sterile spatula to homogenise the reinoculation.

TABLE 3
Microbial challenge test inoculation level

| Challenge | | Inoculum level (cfu/mL) | | |
|---|---|---|---|---|
| test pool | Microorganisms | Day 0 | Day 7 | Day 14 |
| Pool 1 | B. cepacia | 7.1E+08 | 7.8E+08 | 6.1E+08 |
| | P. aeruginosa | | | |
| | Strenotrophomonas | | | |
| | sp | | | |
| Pool 2 | E. cloacae | 5.2E+08 | 4.1E+08 | 6.6E+08 |
| | Klebsiella sp. | | | |

TABLE 4
Microbial challenge test log kill

| | | | Microbial Log reduction | | | |
|---|---|---|---|---|---|---|
| | Sample | Pool | 7 days | 14 days | 21 days | 28 days |
| 1 | Unpreserved | 1 | >5.85 | >5.89 | <2.79 | <2.79 |
| | Conditioner | 2 | 2.62 | <2.61 | <2.82 | <2.82 |
| 2 | 0.5% Itaconic | 1 | >5.85 | >5.89 | >5.79 | >5.79 |
| | acid | 2 | >5.72 | >5.61 | >5.82 | >5.82 |
| 3 | 1% Itaconic acid | 1 | >5.85 | >5.89 | >5.79 | >5.79 |
| | | 2 | >5.72 | >5.61 | >5.82 | >5.82 |
| 4 | 0.5% Lactic acid | 1 | >5.85 | >5.89 | >5.79 | >5.79 |
| | | 2 | 3.73 | 3.70 | <2.82 | <2.82 |
| 5 | 1% Lactic acid | 1 | >5.85 | >5.89 | >5.79 | >5.79 |
| | | 2 | 3.69 | 3.58 | 4.02 | 4.62 |
| 6 | 0.5% Citric acid | 1 | >5.85 | >5.89 | >5.79 | >5.79 |
| | | 2 | 3.20 | 2.28 | <2.82 | 2.72 |
| 7 | 1% Citric acid | 1 | >5.85 | >5.89 | >5.79 | >5.79 |
| | | 2 | 4.67 | 2.86 | <2.82 | 3.69 |

The above results demonstrate the effectiveness of itaconic acid as a preservation chemical for formulations containing cationic surfactants.

1. An aqueous composition comprising itaconic acid or salt thereof and a cationic surfactant.

2. The composition according to claim 1, wherein the level of itaconic acid or salt thereof is from 0.05 to 7 wt % of the total composition.

3. The composition according to claim 1 further comprising an aromatic compound.

4. The composition according to claim 3, wherein the aromatic compound is benzoic acid or a salt thereof.

5. The composition according to claim 1 further comprising a cyclohexane or derivative thereof.

6. The composition according to claim 1 further comprising a terpene and/or thymol.

7. The composition according to claim 1 further comprising an aromatic compound, benzoic acid or salt thereof, a cyclohexane, terpene, and/or thymol, wherein the individual level of aromatic compound, benzoic acid or salt thereof, cyclohexane, terpene and/or thymol is from 0.05 to 3 wt % of the total composition.

8. The composition according to claim 1, wherein the cationic surfactant is selected from the group consisting of cetyltrimethylammonium chloride (CTAC), behentrimonium chloride (BTAC), cetearyl alcohol and stearamidopropyldimethylamine and mixtures thereof.

9. The composition according to claim 1, comprising at least 80 wt % of the total composition of water.

10. The composition according to claim 1, wherein the level of cationic surfactant is from 0.05 to 5 wt % of the total composition.

11. The composition according to claim 1, wherein the weight ratio of cationic surfactant to itaconic acid is from 1:1-1:100.

12. The composition according to claim 1 which is a conditioner.

13. The composition according to claim 1 which is a personal care composition.

14. A method of preserving an aqueous composition comprising cationic surfactant and at least 75 wt % water, by the use of itaconic acid.
